(19) United States
(12) Patent Application Publication
(10) Pub. No.: US 2023/0365721 A1
(43) Pub. Date: Nov. 16, 2023

(54) OAT DERIVED BETA-(1-3)-(1-4)-GLUCAN PREPARATIONS AND THERE USE IN TREATING CANCER

(71) Applicant: CASE WESTERN RESERVE UNIVERSITY, Cleveland, OH (US)

(72) Inventor: Mei ZHANG, Cleveland, OH (US)

(21) Appl. No.: 18/028,627

(22) PCT Filed: Sep. 27, 2021
(86) PCT No.: PCT/US2021/052213

§ 371 Date: Mar. 27, 2023

Related U.S. Application Data
(60) Provisional application No. 63/083,283, filed on Sep. 25, 2020.

(51) Int. Cl. C08B 37/00; A61K 31/716; A61K 36/899

(52) U.S. Cl. CPC C08B 37/0024; C08B 37/0003; A61K 31/716; A61K 36/899; A61K 2236/37; A61K 2236/30

(57) ABSTRACT

A composition comprising an oat derived, salt precipitated β-(1,3)-(1,4) glucan having a molecular weight from about 150 kDa to about 250 kDa, a Mw/Mn ratio of about 1.0 to about 1.25, and a D3/D4 ratio of about 1.5 to less than about 2.0.

### RELATED APPLICATION

This application claims priority from U.S. Provisional Application No. 63/083,283, filed Sep. 25, 2020, the subject matter of which are incorporated herein by reference in their entirety.

### BACKGROUND

Cancer immunotherapy achieves immune-mediated control of tumor growth and metastasis by mounting tumor-reactive T cell responses. Although immunotherapy holds great promise for cancer treatment, its clinical success has so far been limited. Increasingly, studies have demonstrated that cancer cells exploit multiple mechanisms to create an immunosuppressive environment that enable them to escape immune destruction. Therefore, overcoming immunosuppressive mechanisms and induction of durable antitumor immunity using novel immune modulators are essential goals of cancer immunotherapy.

It has previously been demonstrated that β-glucan molecules can be exploited as immune modulators for generating antitumor immune responses, which is based on their ability to integrate innate and adaptive immune components. β-glucans (or polysaccharides) are major cell wall components of microbes. Their carbohydrate structures can be recognized as pathogen associated molecular pattern (PMAP) by pattern recognition receptor (PRR) such as dectin-1 and CR3, which are C-type lectin and carbohydrate PRR. Similar to other PRR such as toll-like receptors (TLRs), carbohydrate PRRs are also involved in host defense mechanisms against infection. However, different from TLRs that recognize various PAMPs such as lipopolysaccharide, proteoglycans, DNAs and RNAs, C-type lectins appear to be more specific and mostly recognize carbohydrate structures. Because of the specific recognition, some β-glucans display a capability of stimulating host immune responses via priming macrophage, neutrophil and granulocytes through dectin-1 and/or complement receptor 3 (CR3). β-glucan mediated action on these receptors can further trigger natural killer (NK) cells, dendritic cells (DCs) and T cells to respond to tumor targets. The glucan molecule-mediated immunomodulation has been attributed to its efficient modulatory function during pathogen recognition and antigen presentation. More importantly, studies on soluble β-glucan have demonstrated that the β-glucan-bound monocytes and neutrophils can mediate direct binding of these cells to complement 3 opsonized targets such as iC3b-bound tumor cells, which provides a cellular mechanism of β-glucan to target tumor environment.

Therefore, modification of tumor environment using novel glucan-type immune modulator can potentially enhance immunogenicity of tumor.

Emerging data suggest that β-glucans from different resources with different impurities, glycosidic linkage, molecular weight, solubility, and route of administration all exhibit different mechanism of actions and potency of antitumor effect. A current limitation in studies involving β-glucan includes a lack of β-glucan control standard with specific molecular weight and branches available. Most of the β-glucans studies to-date used zymosan, which is a mixture of chitosan, β-glucans and cell wall particles; and the exact immunological actions and signaling pathway induced by β-glucan are still unclear and have to be further defined.

### SUMMARY

Embodiments described herein relate to methods of preparing β-glucan concentrates from organic oat bran with specifically defined purity, structure, Mw, Mw distribution, D3/D4 and potency using a technology that involves salt-based precipitants and that allows for scalable production, and, more particularly, to the use of the β-glucan concentrates with specifically defined purity, structure, Mw, Mw distribution, D3/D4 and potency in treating cancer in a subject in need thereof.

In some embodiments, an oat derived β-(1,3)-(1,4) glucan can be salt precipitated such that the β-(1,3)-(1,4) glucan has a molecular weight from about 150 kDa to about 250 kDa (e.g., about 200 kDa ±15 kDa), a Mw/Mn ratio of about 1.0 to about 1.25, and a D3/D4 ratio of about 1.5 to less than about 2.0. The β-(1,3)-(1,4) glucan composition can include at least about 92%, 93%, 94%, 95%, 96%, 98%, 99% or more by weight of the oat derived, salt precipitated β-(1,3)-(1,4) glucan, less than about 2% by weight protein and less than 0.001% by weight fat, and/or an endotoxin level less than about 5 EU/kg.

Advantageously, the oat derived, salt precipitated β-(1,3)-(1,4) glucan upon administration to a THP1 cell culture can modulate THP1 cells into cells with dendritic cell (DC) like phenotype characterized by upregulated activation markers CD80 CD86, MHC II, and CD11c and increased production of inflammatory cytokines TNF-α and IL-12, and enhanced phagocytosis.

In other embodiments, the oat derived, salt precipitated β-(1,3)-(1,4) glucan can be provided in a pharmaceutical composition with a pharmaceutically acceptable carrier. The pharmaceutical composition can be an injectable or intravenous solution that includes about 0.001 mg/ml to about 5 mg/ml of the oat derived, salt precipitated β-(1,3)-(1,4) glucan.

Other embodiments described herein relate to a method of producing an oat derived, salt precipitated ⊖-(1,3)-(1,4) glucan. The method can include providing an aqueous suspension of β-glucan oat bran concentrate and fractional precipitating an aqueous extract of the aqueous suspension with at least one salt precipitant to obtain an oat derived, precipitated β-(41,3)-(1,4) glucan having a molecular weight from about 150 kDa to about 250 kDa (e.g., about 200 kDa±15 kDa), a Mw/Mn ratio of about 1.0 to about 1.25, and a D3/D4 ratio of about 1.5 to less than about 2.0.

In some embodiments, the β-glucan oat bran concentrate can have a glucan concentration of at least 60% by weight.

In other embodiments, the at least one salt precipitant includes a mixture of salts. In some embodiments, the mixture of salts can include ammonium sulfate.

In some embodiments, the aqueous extract is provided by heating the aqueous suspension of the β-glucan concentrate to about 75° C. to about 90° C. for a duration of time effective dissolve soluble β-glucan in the aqueous suspension, cooling the aqueous suspension, and collecting the aqueous extract of the β-glucan oat bran concentrate from the cooled aqueous suspension.

In some embodiments, the fractional precipitation includes two or more fractional precipitation steps. A first fractional precipitation step can include adding a first salt precipitant to the aqueous extract of the aqueous suspension at a concentration effective to form a first precipitate and a first supernatant and then separating the first supernatant and the first precipitate. The first supernatant has a maximum β-glucan molecular weight less about 230 kDa, less than about 225 kDa, less than about 220 kDa, or less than about 215 kDa.

In some embodiments, a second fractional precipitation step can include adding a second salt precipitant to the separated first supernatant at a concentration effective to form a second precipitate and a second supernatant and then separating the second precipitate and the second supernatant. The second supernatant can have a maximum β-glucan molecular weight less about less than about 175 kDa, less than about 180 kDa, less than about 185 kDa, or less than about 190 kDa.

In some embodiments, the first salt precipitant has the same composition as the second salt precipitant. The first salt precipitant and the second salt precipitant can include a mixture of salts, wherein at least one of the salts in the mixture is ammonium sulphate. The first salt precipitant can be added to the first supernatant at a w/w % that differs from a w/w % at which the second salt precipitant is added to the second supernatant.

In some embodiments, the method can further include forming an aqueous solution of the second precipitate and dialyzing the aqueous solution of the second precipitate to remove residual first salt precipitant and second salt precipitant from the aqueous solution of the second precipitate. An alcohol can then be added to the dialyzed aqueous solution to precipitate β-(1,3)-(1,4) glucan having a molecular weight from about 150 kDa to about 250 kDa, a Mw/Mn ratio of about 1.0 to about 1.25, and a D3/D4 ratio of about 1.5 to less than about 2.0.

### BRIEF DESCRIPTION OF THE FIGURES

FIG. 1 is a schematic illustration of the chemical structure of β-(41,3)-(1,4) glucan.

FIG. 2 illustrates a flow chart showing the steps in the preparation of an oat derived, salt precipitated β-(41,3)-(1,4) glucan in accordance with an embodiment described herein.

FIG. 3 illustrates a flowchart showing the steps in the preparation of starting materials (organic oat brans).

FIG. 4 illustrates a flowchart showing the steps in the preparation of β-glucan concentrate from starting material.

FIG. 5 illustrates a flow chart showing the steps in the preparation of the salt precipitated β-(1,3)-(1,4) glucan.

FIG. 6 illustrates: (A) 13 NMR spectra of β-(1,3)-(1,4) glucan with MW of 20 kDa, 200 kDa, and 500 kDa; and (B) GPC-LS spectra of β-(1,3)-(1,4) glucan with MW 10 kDa, 200 kDa, and 500 kDa.

FIG. 7 illustrates DP3/DP4 characterization of β-(1,3)-(1,4) glucan with MW of 200 kDa. (A) Illustration of the mode of action of lichenase on β-glucan, showing linkages hydrolyzed resulting in the generation of DP3 and DP4. (B) HPAEC-PAD chromatogram of oligosaccharides obtained from β-(1,3)-(1,4) glucan with MW of 200 kDa after lichenase hydrolysis.

### DETAILED DESCRIPTION

While the following terms are believed to be well understood by one of ordinary skill in the art, the following definitions are set forth to facilitate explanation of the presently disclosed subject matter.

The articles “a” and “an” are used herein to refer to one or to more than one (i.e., to at least one) of the grammatical object of the article. By way of example, “an element” means one element or more than one element.

The terms “comprise,” “comprising,” “include,” “including,” “have,” and “having” are used in the inclusive, open sense, meaning that additional elements may be included. The terms “such as”, “e.g.”, as used herein are non-limiting and are for illustrative purposes only. “Including” and “including but not limited to” are used interchangeably. The present invention may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

The term “or” as used herein should be understood to mean “and/or”, unless the context clearly indicates otherwise.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

The term “pharmaceutically acceptable” means suitable for use in contact with the tissues of humans and animals without undue toxicity, irritation, allergic response, and the like, commensurate with a reasonable benefit/risk ratio, and effective for their intended use within the scope of sound medical judgment.

The term “treating” is art-recognized and includes inhibiting a disease, disorder or condition in a subject, e.g., impeding its progress; and relieving the disease, disorder or condition, e.g., causing regression of the disease, disorder and/or condition. Treating the disease or condition includes ameliorating at least one symptom of the particular disease or condition, even if the underlying pathophysiology is not affected.

The term “preventing” is art-recognized and includes stopping a disease, disorder or condition from occurring in a subject, which may be predisposed to the disease, disorder and/or condition but has not yet been diagnosed as having it. Preventing a condition related to a disease includes stopping the condition from occurring after the disease has been diagnosed but before the condition has been diagnosed.

A “patient,” “subject,” or “host” to be treated by the subject method may mean either a human or non-human animal, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder.

The phrase “therapeutically effective amount” or “pharmaceutically effective amount” is an art-recognized term. In certain embodiments, the term refers to an amount of a therapeutic agent that produces some desired effect at a reasonable benefit/risk ratio applicable to any medical treatment. In certain embodiments, the term refers to that amount necessary or sufficient to eliminate, reduce or maintain a target of a particular therapeutic regimen. The effective amount may vary depending on such factors as the disease or condition being treated, the particular targeted constructs being administered, the size of the subject or the severity of the disease or condition. One of ordinary skill in the art may empirically determine the effective amount of a particular compound without necessitating undue experimentation.

The effectiveness of treatment may be measured, for example, by evaluating a reduction in tumor load or decrease in tumor growth in a subject in response to the administration of anticancer agents. The reduction in tumor load may be represent a direct decrease in mass, or it may be measured in terms of tumor growth delay, which is calculated by subtracting the average time for control tumors to grow over to a certain volume from the time required for treated tumors to grow to the same volume.

As used herein, the term “tumor” refers to any neoplastic growth, proliferation or cell mass whether benign or malignant (cancerous), whether a primary site lesion or metastases.

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present on a given atom, and, thus, the description includes structures wherein a non-hydrogen substituent is present and structures wherein a non-hydrogen substituent is not present.

Throughout the description, where compositions are described as having, including, or comprising, specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

All percentages and ratios used herein, unless otherwise indicated, are by weight.

Embodiments described herein relate to methods of preparing β-glucan concentrates from organic oat bran with specifically defined purity, structure, Mw, Mw distribution, D3/D4 and potency using a technology that involves salt-based precipitants and that allows for scalable production, and, more particularly, to the use of the β-glucan concentrates with specifically defined purity, structure, Mw, Mw distribution, D3/D4 and potency in treating cancer in a subject in need thereof.

The chemical structure of β-(1,3)-(1-4) glucan is shown in FIG. 1. The mechanism through which β-glucans exert their immunomodulatory effects can be influenced by the structural differences between different forms of the β-glucans, such as its particulate or soluble nature, tertiary conformation, length of main chain, length of side chains, and frequency of side chains.

In some embodiments, an oat derived β-(1,3)-(1,4) glucan can be salt precipitated such that the β-(1,3)-(1,4) glucan has a molecular weight from about 150 kDa to about 250 kDa, a Mw/Mn ratio of about 1.0 to about 1.25, and a D3/D4 ratio of about 1.5 to less than about 2.0. In some embodiments, the β-(1,3)-(1,4) glucan has a molecular weight from 175 to 225 kDa, from 190 to 210 kDa, or about 200kDa.

The β-(1,3)-(1,4) glucan can be highly purified, with a carbohydrate content over 98%, and substantially free of endotoxin. For example, the β-(1,3)-(1,4) glucan composition can include at least about 92%, 93%, 94%, 95%, 96%, 98%, 99% or more by weight of the oat derived, salt precipitated β-(1,3)-(1,4) glucan, less than about 2% by weight protein and less than about 0.001% by weight fat, and/or an endotoxin levels less than about 5 EU/kg.

Advantageously, the oat derived, salt precipitated β-(1,3)-(1,4) glucan upon administration to a THP1 cell culture can modulate THP1 cells into cells with DC like phenotype characterized by upregulated activation markers CD80 CD86, MHC II, and CD11c, increased production of inflammatory cytokines TNF-cα and IL-12, and enhanced phagocytosis.

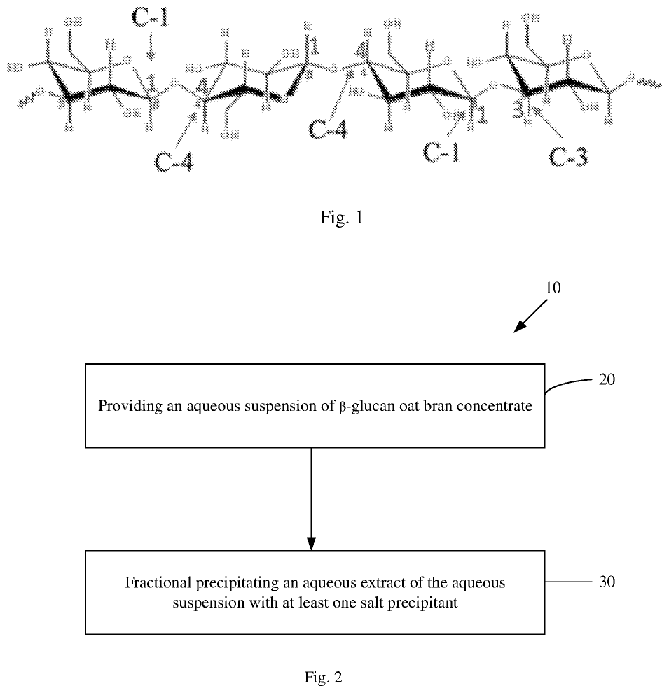

FIG. 2 illustrates a flow chart showing steps of a method 10 of producing the oat derived, salt precipitated β-(1,3)-(1,4) glucan. At step 20, the method 10 includes providing an aqueous suspension of β-glucan oat bran concentrate.

FIG. 4 illustrates a flow diagram showing a method of forming the the β-glucan oat bran concentrate. The β-glucan oat bran concentrate can be formed by adding organic oat bran to distilled water in clean and autoclaved beakers to form an oat bran slurry. The beakers can then be heated under constant stirring in a water bath at about 75° C. to about 90° C. for a duration of time effective to dissolve soluble β-glucan in the aqueous suspension and then cooled to room temperature under constant stirring. The slurry can be centrifuged to form a supernatant and, following centrifugation, the supernatant can be collected and incubated with bacterial α-amylase at room temperature. After incubation, the slurry can be centrifuged and the supernatant formed can be collected. The β-glucan can be precipitated from supernatant by addition of an alcohol, such as isopropanol. β-glucan containing precipitates can be collected by decanting the supernatant and oven-drying the β-glucan precipitate. In some embodiments, the dried precipitate has a β-glucan concentration of at least about 50%, at least about 60%, at least about 70%, or at least about 80% by weight.

The β-glucan precipitate or concentrate can then be suspended in distilled water to form the aqueous suspension of β-glucan oat bran concentrate.

Referring again to FIG. 2, following the step 20 of providing an aqueous suspension of β-glucan oat bran concentrate, at step 30, an aqueous extract of the aqueous suspension is fractionally precipitated with at least one salt precipitant to obtain an oat derived, precipitated β-(1,3)-(1,4) glucan having a molecular weight from about 150 kDa to about 250 kDa, for example, about 175 kDa to about 225 kDa, from about 190 kDa to about 210 kDa, or about 200 kDa, a Mw/Mn ratio of about 1.0 to about 1.25, and a D3/D4 ratio of about 1.5 to less than about 2.0. The aqueous extract can be provided by heating the aqueous suspension of the β-glucan oat bran concentrate to about 75° C. to about 90° C. under constant stirring for a duration of time effective dissolve soluble β-glucan in the aqueous suspension, cooling the aqueous suspension, and collecting the aqueous extract of the β-glucan oat bran concentrate from the cooled aqueous suspension after centrifugation.

The aqueous extract can then be fractionally precipitated with at least one salt precipitant to obtain the oat derived, precipitated β-(1,3)-(1,4) glucan having a molecular weight from about 150 kDa to about 250 kDa, a Mw/Mn ration of about 1.0 to about 1.25, and a D3/D4 ratio of about 1.5 to less than about 2.0.

In some embodiments, the at least one salt precipitant can include a mixture of salts. Examples of salts that can be used to factionally precipitate the aqueous extract to obtain the oat derived, precipitated β-(1,3)-(1,4) glucan include sodium chloride, potassium chloride, ammonium chloride, ammonium bromide, potassium bromide, ammonium bromide, sodium acetate, potassium acetate, ammonium acetate, ammonium sulfate, magnesium sulfate, potassium sulfate, sodium sulfate, sodium magnesium sulfate, sodium iodide, potassium iodide, ammonium thiosulfate, potassium thiosulfate, sodium thiosulfate, and combinations thereof. In some embodiments, the mixture of salts can include ammonium sulfate and at least one, two, three, four or more salts described herein.

In some embodiments, the fractional precipitation used to obtain the oat derived, precipitated β-(1,3)-(1,4) glucan having a molecular weight from about 150 kDa to about 250 kDa, a Mw/Mn ratio of about 1.0 to about 1.25, and a D3/D4 ratio of about 1.5 to less than about 2.0 includes two or more fractional precipitation steps.

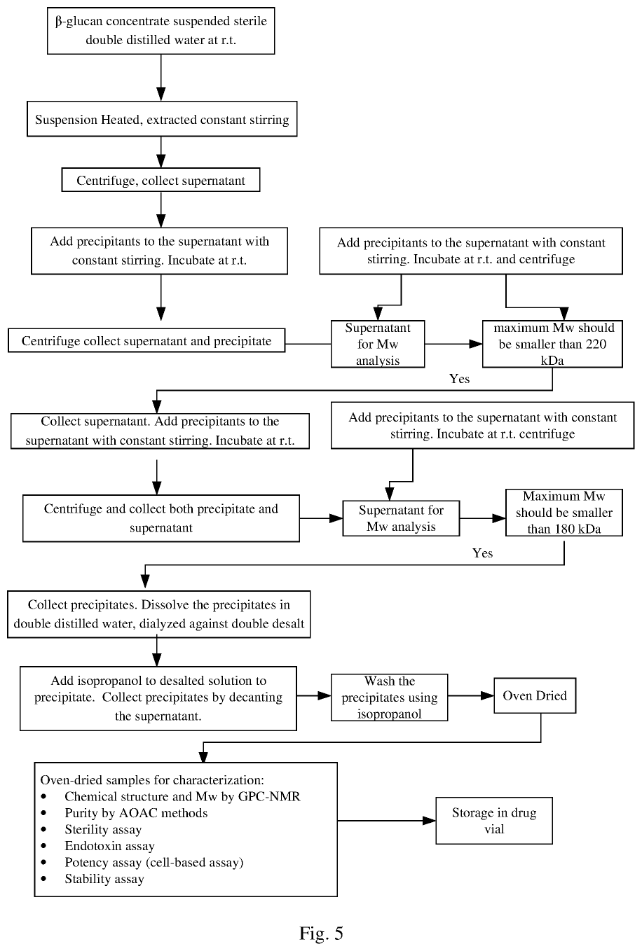

FIG. 5 illustrates a flow chart showing the steps in a method of fractional precipitating the aqueous extract. The method includes a first fractional precipitation step and a second fractional precipitation step.

In some embodiments, the first fractional precipitation step can include adding a first salt precipitant to the aqueous extract of the aqueous suspension at a concentration effective to form a first β-glucan precipitate and a first β-glucan containing supernatant. The first salt precipitant can include a mixture of salts, such as a mixture of one or more salts selected from the group consisting of sodium chloride, potassium chloride, ammonium chloride, ammonium bromide, potassium bromide, ammonium bromide, sodium acetate, potassium acetate, ammonium acetate, ammonium sulfate, magnesium sulfate, potassium sulfate, sodium sulfate, sodiomagnesic sulfate, sodium iodide, potassium iodide, ammonium thiosulfate, potassium thiosulfate, sodium thiosulfate, and combinations thereof. The salt can be added to the aqueous extract at a concentration of at least about 5% (w/w %), at least about 10% (w/w %), at least about 15% (w/w %), at least about 20% (w/w %), at least about 25% (w/w %) or more with constant stirring.

The aqueous extract can then be incubated at room temperature and centrifuged to form the first β-glucan containing supernatant and the first β-glucan precipitate. The first supernatant can be separated from the first precipitate and the molecular weight of the β-glucan in the first supernatant can be measured. If the first supernatant does not have a maximum β-glucan molecular weight less than about 230 kDa, less than about 225 kDa, less than about 220 kDa, or less than about 215 kDa, then additional salt precipitant can be added to the first supernatant until the first supernatant has a maximum β-glucan molecular weight less about 230 kDa, less than about 225 kDa, less than about 220 kDa, or less than about 215 kDa. The first supernatant with a maximum β-glucan molecular weight less about 230 kDa, less than about 225 kDa, less than about 220 kDa, or less than about 215 kDa can be collected and then subjected to the second fractional precipitation step.

The second fractional precipitation step can include adding a second salt precipitant to the collected first supernatant with a maximum β-glucan molecular weight less about 230 kDa, less than about 225 kDa, less than about 220 kDa, or less than about 215 kDa at a concentration effective to form a second β-glucan precipitate and a second β-glucan containing supernatant. The second salt precipitant can include a mixture of salts, such as a mixture of one or more salts selected from the group consisting of sodium chloride, potassium chloride, ammonium chloride, ammonium bromide, potassium bromide, ammonium bromide, sodium acetate, potassium acetate, ammonium acetate, ammonium sulfate, magnesium sulfate, potassium sulfate, sodium sulfate, sodium magnesium sulfate, sodium iodide, potassium iodide, ammonium thiosulfate, potassium thiosulfate, sodium thiosulfate, and combinations thereof. The salt can be added to the first supernatant at a concentration of at least about 5% (w/w %), at least about 10% (w/w %), at least about 15% (w/w %), at least about 20% (w/w %), at least about 25% (w/w %) or more with constant stirring.

The first supernatant can then be incubated at room temperature and centrifuged to form the second β-glucan containing supernatant and the second β-glucan precipitate. The second supernatant can be separated from the second precipitate and the molecular weight of the β-glucan in the second supernatant can be measured. If the second supernatant does not have a maximum β-glucan molecular weight less than about 175 kDa, less than about 180 kDa, less than about 185 kDa, or less than about 190 kDa, then additional salt precipitant can be added to the second supernatant until the second supernatant has a maximum a maximum β-glucan molecular weight less than about 175 kDa, less than about 180 kDa, less than about 185 kDa, or less than about 190 kDa.

The second β-glucan precipitate can have molecular weight from about 150 kDa to about 250 kDa, for example, about 175 kDa to about 225 kDa, about 190 kDa to about 210 kDa, or about 200 kDa and be collected and then desalted to remove impurities from the fractional precipitation processes.

In some embodiments, the first salt precipitant has the same composition as the second salt precipitant. The first salt precipitant and the second salt precipitant include a mixture of salts, wherein at least one of the salts in the mixture is ammonium sulphate and at least one, two, three, four, or more salts described herein. The first salt precipitant can be added to the first supernatant at a w/w % that differs from a w/w % at which the second salt precipitant is added to the second supernatant.

In some embodiments, the method can further include forming an aqueous solution of the second β-glucan precipitate and dialyzing the aqueous solution of the second precipitate to remove residual first salt precipitant and second salt precipitant from the aqueous solution of the second precipitate. An alcohol can then be added to the dialyzed aqueous solution to precipitate β-(1,3)-(1,4) glucan having a molecular weight from about 150 kDa to about 250 kDa, a Mw/Mn ration of about 1.0 to about 1.25, and a D3/D4 ratio of about 1.5 to less than about 2.0.

In some embodiments, the oat derived, salt precipitated β-(1,3)-(1,4) glucan, can be used in a method of treating cancer in a subject in need thereof by administering a therapeutically effective amount of the oat derived, salt precipitated β-(1,3)-(1,4) glucan described herein to the subject. The term “cancer” refers to a proliferative disorder caused or characterized by a proliferation of cells which have lost susceptibility to normal growth control. Cancers of the same tissue type usually originate in the same tissue, and may be divided into different subtypes based on their biological characteristics. Four general categories of cancer are carcinoma (epithelial cell derived), sarcoma (connective tissue or mesodermal derived), leukemia (blood-forming tissue derived) and lymphoma (lymph tissue derived). Over 200 different types of cancers are known, and every organ and tissue of the body can be affected. Specific examples of cancers that do not limit the definition of cancer can include melanoma, leukemia, astrocytoma, glioblastoma, retinoblastoma, lymphoma, glioma, Hodgkin's lymphoma, and chronic lymphocytic leukemia. Examples of organs and tissues that may be affected by various cancers include pancreas, breast, thyroid, ovary, uterus, testis, prostate, pituitary gland, adrenal gland, kidney, stomach, esophagus, rectum, small intestine, colon, liver, gall bladder, head and neck, tongue, mouth, eye and orbit, bone, joints, brain, nervous system, skin, blood, nasopharyngeal tissue, lung, larynx, urinary tract, cervix, vagina, exocrine glands, and endocrine glands. Alternatively, a cancer can be multicentric or of unknown primary site (CUPS). In some embodiments, the method is used to treat a subject having melanoma or osteosarcoma.

In some embodiments, a composition, which includes the oat derived, salt precipitated β-(1,3)-(1,4) glucan, can be administered to a subject to treat metastatic cancer. As used herein, “metastasis” refers to the ability of cells of a cancer (e.g., a primary tumor, or a metastatic tumor) to be transmitted to other locations in the subject (i.e., target organs) and to establish new tumors at such locations. The most common places for the metastases to begin are referred to as the primary cancer, and include the lung, breast, skin, colon, kidney, prostate, pancreas, liver, and cervix. There is a propensity for certain tumors to seed in particular organs. For example, prostate cancer usually metastasizes to the bones. In a similar manner, colon cancer tends to metastasize to the liver. Stomach cancer often metastasizes to the ovary in women. In some embodiments, the oat derived, salt precipitated β-(1,3)-(1,4) glucan is used to treat the metastasis originating from breast cancer, prostate cancer, or lung cancer primary tumors. The cells capable of forming metastatic cancer can be circulating cancer cells within the bloodstream, as opposed to cancer cells present at a fixed location, such as a solid tumor.

In some embodiments, the method is used to treat cancer that has developed immune tolerance Immune tolerance is the state in which cancer cells exhibit decreased immunogenicity or the establishment of an immunosuppressive state within the tumor microenvironment, thereby diminishing the ability of the immune system to attack the cancer cells Immune tolerance is a frequent problem in cancer treatment, in part because the cancer cells have a large number of self-antigens, for which immune tolerance is necessary.

The methods described herein include administration of the oat derived, salt precipitated β-(1,3)-(1,4) glucan, alone, or in combination therapies wherein the subject is also undergoing one or more cancer therapies selected from at least one of surgery, chemotherapy, radiotherapy, thermotherapy, immunotherapy, hormone therapy or laser therapy. Combination therapy can typically include treatment with one or more of chemotherapeutics, tumor-targeting antibodies; adoptive transfer of immune cells (i.e., adoptive immunotherapy); pro-inflammatory cytokines, and the like. Combination therapy can also include conventional therapy, including, but not limited to, antibody administration, vaccine administration, administration of cytotoxic agents, thermoablation, cryoablation, and radioablation.

In some embodiments, the oat derived, salt precipitated β-(1,3)-(1,4) glucan is administered to a subject suffering from a cancer and exposed to, treated with, or undergoing radiotherapy. The combination of oat derived, salt precipitated β-(1,3)-(1,4) glucan with radiotherapy can significantly enhance the subject's anti-cancer immune response. In some embodiments, the radiotherapy can include low-dose (e.g., less than 30 Gray (Gy)) or fractioned radiotherapy that comprises at least one irradiation step wherein the ionizing radiation dose ranges from about 1.5 to 30 Gray (Gy), for example, about 1.5 to about 20 Gray (Gy), typically from about 1.5 to about 15 Gray (Gy).

In the context of a fractioned radiotherapy, the total dose of ionizing radiations is divided into several, smaller doses over a period of several days. This maximizes the effect of radiations on cancer and minimizes the negative side effects on healthy cells. Typical fractionation schemes divide the total dose into 30 units/fractions delivered every weekday over 6 weeks, though current research is considering the benefits of accelerated fractionation (2 deliveries per day and/or deliveries on weekends as well).

The term “Ionizing radiations” refers to highly-energetic particles or waves that can ionize an atom or molecule. Ionizing ability depends on the energy of individual particles or waves, and not on their number. A large flood of particles or waves will not, in the most-common situations, cause ionization if the individual particles or waves are insufficiently energetic. A typical ionizing radiation is a radiation, the energy of which is of at least 1.8 KeV.

In some embodiment, the ionizing radiations dose per irradiation step is selected from 1.8, 2, 2.2, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.5, 6, 6.5, 7, 7.5, 8, 8.5, 9, 9.5, 10, 11, 12, 13, 14, 15, 20, 25 and 30 Gy per fraction treatment. The ionizing radiations dose can also be selected from 1.8, 2, 2.4, 2.5, 3, 3.2, 3.6, 4, 4.5, 5, 5.5, 6, 7, 8, 10, 15, 20, 25 and 30 Gy per fraction treatment, for example from 2, 3, 5, 6, 7, 8, 10, 15, 20, 25 and 30 Gy.

In other embodiments, fractionated radiotherapy can be selected from 25 fractions of 2 Gy (total: 50 Gy), 30 fractions of 2 Gy (total: 60 Gy), 35 fractions of 2 Gy (total: 70 Gy), 40 fractions of 2 Gy (total: 80 Gy), 5 fractions of 3 Gy (total: 15 Gy), 10 fractions of 3 Gy (total: 30 Gy), 15 fractions of 3 Gy (total: 45 Gy), 20 fractions of 3 Gy (total: 60 Gy), 25 fractions of 3 Gy (total: 75 Gy), 3 fractions of 4 Gy (total: 12 Gy), 5 fractions of 4 Gy (total: 20 Gy), 8 fractions of 4 Gy (total: 32 Gy), 10 fractions of 4 Gy (total: 40 Gy), 15 fractions of 4 Gy (total: 60 Gy), 20 fractions of 4 Gy (total: 80 Gy), 2 fractions of 5 Gy (total: 10 Gy), 3 fractions of 5 Gy (total: 15 Gy), 4 fractions of 5 Gy (total: 20 Gy), 5 fractions of 5 Gy (total: 25 Gy), 6 fractions of 5 Gy (total: 30 Gy), 8 fractions of 5 Gy (total: 40 Gy), 10 fractions of 5 Gy (total: 50 Gy), 1 fraction of 6 Gy (total: 6 Gy), 2 fractions of 6 Gy (total: 12 Gy), 3 fractions of 6 Gy (total: 18 Gy), 4 fractions of 6 Gy (total: 24 Gy), 5 fractions of 6 Gy (total: 30 Gy), 6 fractions of 6 Gy (total: 36 Gy), 10 fractions of 6 Gy (total: 60 Gy), 1 fraction of 7 Gy (total: 7 Gy), 2 fractions of 7 Gy (total: 14 Gy), 3 fractions of 7 Gy (total: 21 Gy), 4 fractions of 7 Gy (total: 28 Gy), 5 fractions of 7 Gy (total: 35 Gy), 1 fraction of 8 Gy (total: 8 Gy), 2 fractions of 8 Gy (total: 16 Gy), 3 fractions of 8 Gy (total: 24 Gy), 4 fractions of 8 Gy (total: 32 Gy), 5 fractions of 8 Gy (total: 40 Gy), 1 fraction of 9 Gy (total: 9 Gy), 2 fractions of 9 Gy (total: 18 Gy), 3 fractions of 9 Gy (total: 27 Gy), 4 fractions of 9 Gy (total: 36 Gy), 5 fractions of 9 Gy (total: 45 Gy), 1 fraction of 10 Gy (total: 10 Gy), 2 fractions of 10 Gy (total: 20 Gy), 3 fractions of 10 Gy (total: 30 Gy), 4 fractions of 10 Gy (total: 40 Gy), 1 fraction of 15 Gy (total: 15 Gy), 2 fractions of 15 Gy (total: 30 Gy), 3 fractions of 15 Gy (total: 45 Gy), 4 fractions of 15 Gy (total: 60 Gy), 1 fraction of 20 Gy (total: 20 Gy), 2 fractions of 20 Gy (total: 40 Gy), 3 fractions of 20 Gy (total: 60 Gy), 1 fraction of 25 Gy (total: 25 Gy), 2 fractions of 25 Gy (total: 50 Gy), 3 fractions of 25 Gy (total: 75 Gy), 1 fraction of 30 Gy (total: 30 Gy), and 2 fractions of 30 Gy (total: 60 Gy).

In some embodiments, the subject can be a subject suffering from metastatic cancer and undergoing a palliative radiotherapy, a subject suffering from metastatic cancer for whom radiotherapy has been abandoned, or a subject suffering from a cancer which is not treated by radiotherapy, and the fractionated radiotherapy is selected from 1 fraction of 6 Gy (total: 6 Gy), 2 fractions of 6 Gy (total: 12 Gy), 3 fractions of 6 Gy (total: 18 Gy), 4 fractions of 6 Gy (total: 24 Gy), 5 fractions of 6 Gy (total: 30 Gy), 1 fraction of 7 Gy (total: 7 Gy), 2 fractions of 7 Gy (total: 14 Gy), 3 fractions of 7 Gy (total: 21 Gy), 4 fractions of 7 Gy (total: 28 Gy), 1 fraction of 8 Gy (total: 8 Gy), 2 fractions of 8 Gy (total: 16 Gy), 3 fractions of 8 Gy (total: 24 Gy), 4 fractions of 8 Gy (total: 32 Gy), 1 fraction of 9 Gy (total: 9 Gy), 2 fractions of 9 Gy (total: 18 Gy), 3 fractions of 9 Gy (total: 27 Gy), 1 fraction of 10 Gy (total: 10 Gy), 2 fractions of 10 Gy (total: 20 Gy), 3 fractions of 10 Gy (total: 30 Gy), 1 fraction of 15 Gy (total: 15 Gy), 2 fractions of 15 Gy (total: 30 Gy), 1 fraction of 20 Gy (total: 20 Gy), 2 fractions of 20 Gy (total: 40 Gy), 1 fraction of 25 Gy (total: 25 Gy) and 1 fraction of 30 Gy (total: 30 Gy).

In some embodiments, the oat derived, salt precipitated β-(1,3)-(1,4) glucan, can be co-administered with another pharmaceutical agent. The two components may be co-administered simultaneously or sequentially. Simultaneously co-administered components may be provided in one or more pharmaceutical compositions. Sequential co-administration of two or more components includes cases in which the components are administered so that both components are simultaneously bioavailable after both are administered. Regardless of whether the components are co-administered simultaneously or sequentially, the components may be co-administered at a single site or at different sites.

In some embodiments, the pharmaceutical agent co-administered with the oat derived, salt precipitated β-(1,3)-(1,4) glucan can include a chemotherapeutic agent. Examples of chemotherapeutic agents that can be co-administered with the oat derived, salt precipitated β-(1,3)-(1,4) glucan, for cancer treatment include alkylating agents, antimetabolites, natural products, hormones and antagonists, and miscellaneous agents. Examples of alkylating agents include nitrogen mustards such as mechlorethamine, cyclophosphamide, ifosfamide, melphalan (L-sarcolysin) and chlorambucil; ethylenimines and methylmelamines such as hexamethylmelamine and thiotepa; alkyl sulfonates such as busulfan; nitrosoureas such as carmustine (BCNU), semustine (methyl-CCNU), lomustine (CCNU) and streptozocin (streptozotocin); DNA synthesis antagonists such as estramustine phosphate; and triazines such as dacarbazine (DTIC, dimethyl-triazenoimidazolecarboxamide) and temozolomide. Examples of antimetabolites include folic acid analogs such as methotrexate (amethopterin); pyrimidine analogs such as fluorouracin (5-fluorouracil, 5-FU, 5FU), floxuridine (fluorodeoxyuridine, FUdR), cytarabine (cytosine arabinoside) and gemcitabine; purine analogs such as mercaptopurine (6-niercaptopurine, 6-MP), thioguanine (6-thioguanine, TG) and pentostatin (2′-deoxycoformycin, deoxycoformycin), cladribine and fludarabine; and topoisomerase inhibitors such as amsacrine. Examples of natural products include vinca alkaloids such as vinblastine (VLB) and vincristine; taxanes such as paclitaxel (Abraxane) and docetaxel (Taxotere); epipodophyllotoxins such as etoposide and teniposide; camptothecins such as topotecan and irinotecan; antibiotics such as dactinomycin (actinomycin D), daunorubicin (daunomycin, rubidomycin), doxorubicin, bleomycin, mitomycin (mitomycin C), idarubicin, epirubicin; enzymes such as L-asparaginase; and biological response modifiers such as interferon alpha and interleukin 2. Examples of hormones and antagonists include luteinizing releasing hormone agonists such as buserelin; adrenocorticosteroids such as prednisone and related preparations; progestins such as hydroxyprogesterone caproate, medroxyprogesterone acetate and megestrol acetate; estrogens such as diethylstilbestrol and ethinyl estradiol and related preparations; estrogen antagonists such as tamoxifen and anastrozole; androgens such as testosterone propionate and fluoxymesterone and related preparations; androgen antagonists such as flutamide and bicalutamide; and gonadotropin-releasing hormone analogs such as leuprolide. Examples of miscellaneous agents include thalidomide; platinum coordination complexes such as cisplatin (czs-DDP), oxaliplatin and carboplatin; anthracenediones such as mitoxantrone; substituted ureas such as hydroxyurea; methylhydrazine derivatives such as procarbazine (N-methylhydrazine, MIH); adrenocortical suppressants such as mitotane (o, p′-DDD) and aminoglutethimide; RXR agonists such as bexarotene; and tyrosine kinase inhibitors such as imatinib.

In some embodiments, the cancer can be further treated with adoptive immunotherapy. Adoptive immunotherapy is a form of immunotherapy in which lymphocytes taken from a patient are grown in large numbers, stimulated, activated, and infused back into the patient. Adoptive immunotherapy can use a variety of different immune cells, including lymphokine-activated killer (LAK) cells, tumor-infiltrating lymphocytes (TILs), and immune effector cells such as T-lymphocytes (e.g., cytokine activated T-cells). See Ruella M, Kalos M., Immunol Rev., 257(1):14-38 (2014). For example, in some embodiments, cancer treatment using β-glucan can be combined with adoptive transfer of T-lymphocytes (e.g., tumor draining lymph node T-lymphocytes).

In some embodiments, the administration of the oat derived, salt precipitated β-(1,3)-(1,4) glucan, is combined with substances that activate T-cells, or inflammatory cytokines. Examples of substances that activate T-cells include IL-2, Opdivo (nivozumab, PD-1 inhibitor, by Bristol-Myers); Keytruda (pembrolizumab, PD-1 inhibitor, by Merck & Co.), Tecentriq (atezolizumab, PD-L1 inhibitor, by Genentech), Imfinzi (durvalumab, PD-L1 inhibitor, by AstraZeneca), or Bavencio (Avelumab, PD-L1 inhibitor, by EMD Serono Inc.). Examples of inflammatory cytokines include CCL3, CC14, TNF-α, and interferon-γ. A variety of small molecule inhibitors of the TGF-β type 1 receptor can also be used to stimulate inflammation.

In some embodiments, the subject treated with the oat derived, salt precipitated β-(1,3)-(1,4) glucan and optionally, one or more other therapies described herein, such as radiotherapy, can be a pediatric subject having any type of cancer. For example, a pediatric subject is a subject from the day of its birth (e.g., 0 days of age) to about 21 years of age. In some embodiments, a pediatric subject is a subject from the day of its birth (e.g., 0 days of age) to about 18 years of age. In some embodiments, a pediatric subject is a subject from about 1 day of age to about 21 years of age. In some embodiments, a pediatric subject is a subject from about 1 day of age to about 18 years of age.

In some embodiments, the subject treated with the oat derived, salt precipitated β-(1,3)-(1,4) glucan and optionally one or more other therapies described herein can have a pediatric cancer. Examples of pediatric cancers include adrenocortical carcinoma, astrocytoma, atypical teratoid rhabdoid tumor, brain tumors, chondroblastoma, choroid plexus tumor, craniopharyngioma, desmoid tumor, dysembryplastic neuroepithelial tumor (DNT), ependymoma, fibrosarcoma, germ cell tumor of the brain, glioblastoma multiforme, diffuse pontine glioma, low grade glioma, gliomatosis cerebri, hepatoblastoma, histiocytosis, kidney tumor, acute lymphoblastic leukemia (ALL), acute myeloid leukemia (AML), chronic myelogenous leukemia (CML), liposarcoma, liver cancer, Burkitt lymphoma, Hodgkin lymphoma, non-Hodgkin lymphoma, malignant fibrous histiocytoma, melanoma, myelodysplastic syndrome, nephroblastoma, neuroblastoma, neurofibrosarcoma, osteosarcoma, pilocytic astrocytoma, retinoblastoma, rhabdoid tumor of the kidney, rhabdomyosarcoma, Ewing sarcoma, soft tissue sarcoma, synovial sarcoma, spinal cord tumor and Wilm's tumor.

In some embodiments, a cancer is Ewing's sarcoma, osteosarcoma, rhabdomyosarcoma (RMS) such as embryonal rhabdomyosarcoma (ERS), a CNS tumor, or neuroblastoma. In some embodiments, a cancer is a CNS tumor.

In some embodiments, a cancer is Ewing's sarcoma (ES), osteosarcoma (OS), rhabdomyosarcoma (RMS), neuroblastoma (NB), medulloblastoma (MB), high-grade glioma (HGG), or adrenocortical carcinoma (ACC).

Once disease is established and a treatment protocol is initiated, evaluation of the cancer can be repeated on a regular basis to evaluate whether the cancer cells in the subject begin to show resistance to the therapy. The results obtained from successive assays may be used to show the efficacy of treatment over a period ranging from several days to months. Accordingly, other embodiments described herein are directed to methods for monitoring a therapeutic regimen for treating a subject having cancer by oat derived, salt precipitated β-(1,3)-(1,4) glucan administration. A comparison of the total cell number (and/or blood cell count) prior to and during therapy indicates the efficacy of the therapy. Likewise, a comparison of the severity of the side effects of chemotherapy or radiotherapy prior to and during oat derived, salt precipitated β-(1,3)-(1,4) glucan therapy indicates the efficacy of oat derived, salt precipitated β-(1,3)-(1,4) glucan therapy. Therefore, one skilled in the art can be able to recognize and adjust the therapeutic approach as needed.

In some embodiments, biomarker levels may also be used as a factor for determining administration of the oat derived, salt precipitated β-(1,3)-(1,4) glucan to a subject, including route and/or intervals. Biomarker levels may be used in combination with other factors such as the nature, severity of the cancer and extent of the subject's condition, and/or to identify an appropriate treatment regimen.

In some embodiments, a subject receives treatment independent of biomarker status. In some embodiments, a subject receives treatment without determination of biomarker status. In some embodiments, a subject receives treatment prior to determination of biomarker status.

As used herein, a “biomarker” or “marker” can include but is limited to a gene, mRNA, protein, or cell phenotype, which can be altered, wherein said alteration is associated with cancer. The alteration can be in amount, structure, and/or activity in a cancer tissue or cancer cell, as compared to its amount, structure, and/or activity, in a normal or healthy tissue or cell (e.g., a control), and is associated with a disease state, such as cancer. For example, a marker associated with cancer, or predictive of responsiveness to anti-cancer therapeutics, can have an altered nucleotide sequence, amino acid sequence, chromosomal translocation, intra-chromosomal inversion, copy number, expression level, protein level, protein activity, epigenetic modification (e.g., methylation or acetylation status, or post-translational modification, in a cancer tissue or cancer cell as compared to a normal, healthy tissue or cell. Furthermore, a “marker” includes a molecule whose structure is altered, e.g., mutated (contains a mutation), e.g., differs from the wild-type sequence at the nucleotide or amino acid level, e.g., by substitution, deletion, or insertion, when present in a tissue or cell associated with a cancer.

In some embodiments, biomarkers associated with antitumor efficacy induced by treatment of a subject having cancer with the oat derived, salt precipitated β-(1,3)-(1,4) glucan can include an increase in a measured level(s) of tumor related factors compared to a control level(s). The increase in measured level(s) of tumor related factors can include an increase in the measured level of at least one of IFN-y expression, TNF-α expression, or PD-L1 expression compared to a control level or a measured level of tumor related factors from a biological sample of the subject prior to administration of the oat derived, salt precipitated β-(1,3)-(1,4) glucan.

In other embodiments, biomarkers associated with antitumor efficacy induced by treatment of a subject having cancer with the oat derived, salt precipitated β-(1,3)-(1,4) glucan can include changes within the tumor microenvironment. Changes within the tumor microenvironment associated with antitumor efficacy include a gene expression signature indicative of inflammation and/or a change in the immune landscape of the tumor microenvironment. The change in immune landscape can include an increase in the number or frequency of CD3/CD4/CD8 cells, an increase in CD11b⁺C11c⁺ cells, or an increase in the number or frequency of M1 macrophages compared to a control number or frequency or a measured frequency or number of cells in the tumor microenvironment of the subject prior to administration of the oat derived, salt precipitated β-(1,3)-(1,4) glucan.

In some embodiments, biomarkers associated with antitumor efficacy induced by treatment with the oat derived, salt precipitated β-(1,3)-(1,4) glucan can include measured increases in the circulating levels of at least one of IFN-γ, TNF-α, CD11b+CCR2+ inflammatory monocytes, or MHC II expression by circulating leukocytes compared to a control level or frequency or a measured frequency or level in the circulation of the subject prior to administration of the oat derived, salt precipitated β-(1,3)-(1,4) glucan.

In other embodiments, biomarkers associated with antitumor efficacy induced by treatment with the oat derived, salt precipitated β-(1,3)-(1,4) glucan can include measured increases of macrophage/dendritic progenitor cells and common dendritic progenitor cells in the bone marrow of the subject compared to a control level or frequency or a measured frequency or level in the bone marrow of the subject prior to administration of the oat derived, salt precipitated β-(1,3)-(1,4) glucan.

A summary of the biomarkers for antitumor efficacy induced by administration of the oat derived, salt precipitated β-(1,3)-(1,4) glucan is provided in Table 1.

TABLE 1
Potential biomarkers for antitumor efficacy induced
by oat derived, salt precipitated β-3,4 glucan

| Tissues | Factors | Favorable | Unfavorable |
|---|---|---|---|
| Tumor | IFN-gamma gene | High | Low |
| related | signature | | |
| factors | Tnf-alpha gene | High | Low |
| | expression | | |
| | PD-L1 expression | High | Low |
| Tumor | Gene expression | Inflamed | non- |
| micro- | signature | | inflamed |
| envionment | Immune landscape | CD3/CD4/CD8 | MDSC |
| | | CD11b⁺CD11c⁺cell | M2 |
| | | frequency | macrophages |
| | | M1 macrophages | |
| Circulation | Plasma level of IFN- | high | low |
| | gamma, TNF-alfa and | | |
| | CCL2 | | |
| | Frequency of | high | low |
| | CD11b⁺CCR2⁺ | | |
| | inflammatory | | |
| | monocytes | | |
| | Overall expression of | high | low |
| | MHC II by circulating | | |
| | leukocytes | | |
| Bone | Gene signature for | High | Low |
| marrow | MDPs | | |
| | (macrophage/dendritic | | |
| | cell progenitor cells) | | |
| | and CDP (common | | |
| | dendritic cell | | |
| | progenitors) | | |
| | Cell frequency of | High | Low |
| | MDP and CDP | | |

In other embodiments, a composition that includes the oat derived, salt precipitated β-(1,3)-(1,4) glucan can be used in a method of immunostimulation that includes administering an effective amount of an oat derived, salt precipitated β-(1,3)-(1,4) glucan to a subject “Immunostimulation” as used herein refers to stimulation of the immune system by inducing activation or increasing activity of any of its components. In some embodiments, immunostimulation includes stimulation of an inflammatory response. In other embodiments, immunostimulation includes stimulation of the cellular immune system. For example, in some embodiments, immunostimulation includes macrophage activation, while in further embodiments the immunostimulation includes T-cell activation.

Immunostimulation can be beneficial for a subject suffering from suppressed immunity. Impairment of any of the major components of the immune system (T-cells, B-cells phagocytes, complement) may result in suppressed immunity Immune defects can arise from intrinsic or heritable defects of lymphoid elements, failure of normal cellular differentiation, diseases such as cancer or viral infection, or other acquired causes. Clinical impairment of immunity is expressed as a marked susceptibility to opportunistic and pathogenic organisms which are difficult to control and by an increased risk of malignancy, allergy and autoimmune disease. In some embodiments, the method of immunostimulation is used to stimulate the immune system of a subject that has cancer, while in further embodiments the subject has cancer that has developed immune tolerance. The method of immunostimulation can include administration of any of the types of oat derived, salt precipitated β-(1,3)-(1,4) glucan described herein.

The oat derived, salt precipitated β-(1,3)-(1,4) glucan, and any additional pharmaceutical agents (e.g., anticancer agents), or a combination thereof, may be formulated into a pharmaceutical composition. In some embodiments, the oat derived, salt precipitated β-(1,3)-(1,4) glucan and the pharmaceutical agent may be provided in a single formulation. In other embodiments, the oat derived, salt precipitated β-(1,3)-(1,4) glucan and the pharmaceutical agent may be provided in separate formulations. A pharmaceutical composition may be formulated in a variety of and/or a plurality forms adapted to one or more preferred routes of administration. Thus, a pharmaceutical composition can be administered via one or more known routes including, for example, oral, parenteral (e.g., intradermal, transcutaneous, subcutaneous, intramuscular, intravenous, intraperitoneal, etc.), or topical (e.g., intranasal, intrapulmonary, intramammary, intravaginal, intrauterine, intradermal, transcutaneous, rectally, etc.). A pharmaceutical composition, or a portion thereof, also can be administered via a sustained or delayed release.

The oat derived, salt precipitated β-(1,3)-(1,4) glucan, the pharmaceutical agent, and/or the combination of both components may be provided in any suitable form including but not limited to a solution, a suspension, an emulsion, a spray, an aerosol, or any form of mixture. A pharmaceutical composition comprising the oat derived, salt precipitated β-(1,3)-(1,4) glucan, the pharmaceutical agent, and/or the combination of both components may be delivered in formulation with any pharmaceutically acceptable excipient, carrier, or vehicle. Pharmaceutically acceptable carriers useful for formulating the oat derived, salt precipitated β-(1,3)-(1,4) glucan for administration to a subject are well known in the art and include, for example, aqueous solutions such as water or physiologically buffered saline or other solvents or vehicles such as glycols, glycerol, oils such as olive oil or injectable organic esters. A pharmaceutically acceptable carrier can contain physiologically acceptable compounds that act, for example, to stabilize or to increase the absorption of the oat derived, salt precipitated β-(1,3)-(1,4) glucan. Such physiologically acceptable compounds include, for example, carbohydrates, such as glucose, sucrose or dextrans, antioxidants, such as ascorbic acid or glutathione, chelating agents, low molecular weight proteins or other stabilizers or excipients. One skilled in the art would know that the choice of a pharmaceutically acceptable carrier, including a physiologically acceptable compound, depends, for example, on the physico-chemical characteristics of the therapeutic agent and on the route of administration of the composition, which can be, for example, orally or parenterally such as intravenously, and by injection, intubation, or other such method known in the art. The pharmaceutical composition also can contain a second (or more) compound(s) such as a diagnostic reagent, nutritional substance, toxin, or therapeutic agent, for example, a cancer chemotherapeutic agent and/or vitamin(s).

A formulation may be conveniently presented in unit dosage form and may be prepared by methods well known in the art of pharmacy. Methods of preparing a composition with a pharmaceutically acceptable carrier include the step of bringing the oat derived, salt precipitated β-(1,3)-(1,4) glucan and/or the pharmaceutical agent into association with a carrier that constitutes one or more accessory ingredients. In general, a formulation may be prepared by uniformly and/or intimately bringing the active compound into association with a liquid carrier, a finely divided solid carrier, or both, and then, if necessary, shaping the product into the desired formulations.

The pharmaceutical composition may be formulated into various dosage forms as discussed above and then administered through various routes including an oral, inhalational, transdermal, subcutaneous, intravenous or intramuscular route. The dosage can be a pharmaceutically or therapeutically effective amount.

Therapeutically effective dosage amounts of the oat derived, salt precipitated β-(1,3)-(1,4) glucan may be present in varying amounts in various embodiments. For example, in some embodiments, a therapeutically effective amount of the oat derived, salt precipitated β-(1,3)-(1,4) glucan may be an amount ranging from about 10-1000 mg (e.g., about 20 mg-1,000 mg, 30 mg-1,000 mg, 40 mg-1,000 mg, 50 mg-1,000 mg, 60 mg-1,000 mg, 70 mg-1,000 mg, 80 mg-1,000 mg, 90 mg-1,000 mg, about 10-900 mg, 10-800 mg, 10-700 mg, 10-600 mg, 10-500 mg, 100-1000 mg, 100-900 mg, 100-800 mg, 100-700 mg, 100-600 mg, 100-500 mg, 100-400 mg, 100-300 mg, 200-1000 mg, 200-900 mg, 200-800 mg, 200-700 mg, 200-600 mg, 200-500 mg, 200-400 mg, 300-1000 mg, 300-900 mg, 300-800 mg, 300-700 mg, 300-600 mg, 300-500 mg, 400 mg-1,000 mg, 500 mg-1,000 mg, 100 mg-900 mg, 200 mg-800 mg, 300 mg-700 mg, 400 mg-700 mg, and 500 mg-600 mg). In some embodiments, the oat derived, salt precipitated β-(1,3)-(1,4) glucan is present in an amount of or greater than about 10 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg. In some embodiments, the oat derived, salt precipitated β-(1,3)-(1,4) glucan is present in an amount of or less than about 1000 mg, 950 mg, 900 mg, 850 mg, 800 mg, 750 mg, 700 mg, 650 mg, 600 mg, 550 mg, 500 mg, 450 mg, 400 mg, 350 mg, 300 mg, 250 mg, 200 mg, 150 mg, or 100 mg.

In other embodiments, a therapeutically effective dosage amount may be, for example, about 0.001 mg/kg weight to 500 mg/kg weight, e.g., from about 0.001 mg/kg weight to 400 mg/kg weight, from about 0.001 mg/kg weight to 300 mg/kg weight, from about 0.001 mg/kg weight to 200 mg/kg weight, from about 0.001 mg/kg weight to 100 mg/kg weight, from about 0.001 mg/kg weight to 90 mg/kg weight, from about 0.001 mg/kg weight to 80 mg/kg weight, from about 0.001 mg/kg weight to 70 mg/kg weight, from about 0.001 mg/kg weight to 60 mg/kg weight, from about 0.001 mg/kg weight to 50 mg/kg weight, from about 0.001 mg/kg weight to 40 mg/kg weight, from about 0.001 mg/kg weight to 30 mg/kg weight, from about 0.001 mg/kg weight to 25 mg/kg weight, from about 0.001 mg/kg weight to 20 mg/kg weight, from about 0.001 mg/kg weight to 15 mg/kg weight, from about 0.001 mg/kg weight to 10 mg/kg weight.

In still other embodiments, a therapeutically effective dosage amount may be, for example, about 0.0001 mg/kg weight to 0.1 mg/kg weight, e.g. from about 0.0001 mg/kg weight to 0.09 mg/kg weight, from about 0.0001 mg/kg weight to 0.08 mg/kg weight, from about 0.0001 mg/kg weight to 0.07 mg/kg weight, from about 0.0001 mg/kg weight to 0.06 mg/kg weight, from about 0.0001 mg/kg weight to 0.05 mg/kg weight, from about 0.0001 mg/kg weight to about 0.04 mg/kg weight, from about 0.0001 mg/kg weight to 0.03 mg/kg weight, from about 0.0001 mg/kg weight to 0.02 mg/kg weight, from about 0.0001 mg/kg weight to 0.019 mg/kg weight, from about 0.0001 mg/kg weight to 0.018 mg/kg weight, from about 0.0001 mg/kg weight to 0.017 mg/kg weight, from about 0.0001 mg/kg weight to 0.016 mg/kg weight, from about 0.0001 mg/kg weight to 0.015 mg/kg weight, from about 0.0001 mg/kg weight to 0.014 mg/kg weight, from about 0.0001 mg/kg weight to 0.013 mg/kg weight, from about 0.0001 mg/kg weight to 0.012 mg/kg weight, from about 0.0001 mg/kg weight to 0.011 mg/kg weight, from about 0.0001 mg/kg weight to 0.01 mg/kg weight, from about 0.0001 mg/kg weight to 0.009 mg/kg weight, from about 0.0001 mg/kg weight to 0.008 mg/kg weight, from about 0.0001 mg/kg weight to 0.007 mg/kg weight, from about 0.0001 mg/kg weight to 0.006 mg/kg weight, from about 0.0001 mg/kg weight to 0.005 mg/kg weight, from about 0.0001 mg/kg weight to 0.004 mg/kg weight, from about 0.0001 mg/kg weight to 0.003 mg/kg weight, from about 0.0001 mg/kg weight to 0.002 mg/kg weight. In some embodiments, the therapeutically effective dose may be 0.0001 mg/kg weight, 0.0002 mg/kg weight, 0.0003 mg/kg weight, 0.0004 mg/kg weight, 0.0005 mg/kg weight, 0.0006 mg/kg weight, 0.0007 mg/kg weight, 0.0008 mg/kg weight, 0.0009 mg/kg weight, 0.001 mg/kg weight, 0.002 mg/kg weight, 0.003 mg/kg weight, 0.004 mg/kg weight, 0.005 mg/kg weight, 0.006 mg/kg weight, 0.007 mg/kg weight, 0.008 mg/kg weight, 0.009 mg/kg weight, 0.01 mg/kg weight, 0.02 mg/kg weight, 0.03 mg/kg weight, 0.04 mg/kg weight, 0.05 mg/kg weight, 0.06 mg/kg weight, 0.07 mg/kg weight, 0.08 mg/kg weight, 0.09 mg/kg weight, or 0.1 mg/kg weight. The effective dose for a particular individual can be varied (e.g., increased or decreased) over time, depending on the needs of the individual.

In some embodiments, a therapeutically effective dosage may be a dosage of 10 μg/kg/day, 50 μg/kg/day, 100 μg/kg/day, 250 μg/kg/day, 500 μg/kg/day, 1000 μg/kg/day or more. In various embodiments, the amount of the oat derived, salt precipitated β-(1,3)-(1,4) glucan is sufficient to provide a dosage to a patient of between 0.01 μg/kg and 10 μg/kg; 0.1 μg/kg and 5 μg/kg; 0.1 μg/kg and 1000 μg/kg; 0.1 μg/kg and 900 μg/kg; 0.1 μg/kg and 900 μg/kg; 0.1 μg/kg and 800 μg/kg; 0.1 μg/kg and 700 μg/kg; 0.1 μg/kg and 600 μg/kg; 0.1 μg/kg and 500 μg/kg; or 0.1 μg/kg and 400 μg/kg.

Particular doses or amounts to be administered may vary, for example, depending on the nature and/or extent of the desired outcome, on particulars of route and/or timing of administration, and/or on one or more characteristics (e.g., weight, age, personal history, genetic characteristic, lifestyle parameter, severity of cardiac defect and/or level of risk of cardiac defect, etc., or combinations thereof). Such doses or amounts can be determined by those of ordinary skill. In some embodiments, an appropriate dose or amount is determined in accordance with standard clinical techniques. For example, in some embodiments, an appropriate dose or amount is a dose or amount sufficient to reduce cancer volume by at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100% or more. Alternatively or additionally, in some embodiments, an appropriate dose or amount is determined through use of one or more in vitro or in vivo assays to help identify desirable or optimal dosage ranges or amounts to be administered.

Alternatively, the dose may be calculated using actual body weight obtained just prior to the beginning of a treatment course. For the dosages calculated in this way, body surface area (m²) is calculated prior to the beginning of the treatment course using the Dubois method: m²=(wt kg^{0.425}×height cm^{0.725})×0.007184. In some embodiments, therefore, the method can include administering sufficient oat derived, salt precipitated β-(1,3)-(1,4) glucan to provide a dose of, for example, from about 0.01 mg/m² to about 10 mg/m².

In some embodiments, a composition or formulation that includes the oat derived, salt precipitated β-(1,3)-(1,4) glucan can be administered to a subject in need thereof once. In other embodiments, a composition or formulation that includes the oat derived, salt precipitated β-(1,3)-(1,4) glucan can be administered to a subject in need thereof more than once. In some embodiments, a first administration of a composition disclosed herein is followed by a second administration of a composition disclosed herein. In some embodiments, a first administration of a composition disclosed herein is followed by a second and third administration of a composition disclosed herein. In some embodiments, a first administration of a composition disclosed herein is followed by a second, third, and fourth administration of a composition disclosed herein. In some embodiments, a first administration of a composition disclosed herein is followed by a second, third, fourth, and fifth administration of a composition disclosed herein. In some embodiments, a first administration of a composition disclosed herein is followed by a drug holiday.

The number of times a composition is administered to a subject in need thereof depends on the discretion of a medical professional, the disorder, the severity of the disorder, and the subject's response to the formulation.

In some embodiments, the composition is administered at predetermined time intervals over an extended period of time. In some embodiments, a composition including the oat derived, salt precipitated β-(1,3)-(1,4) glucan can be administered once every day. In some embodiments, a composition including the oat derived, salt precipitated β-(1,3)-(1,4) glucan can be administered every other day. In some embodiments, a composition including the oat derived, salt precipitated β-(1,3)-(1,4) glucan can be administered over 1 week, 2 weeks, 1 month, 2 months, 3 months, 6 months, 1 year, 2 years, 3 years, 4 years, 5 years, 6 years, 7 years, 8 years, 9 years, 10 years, 11 years, or 12-15 years.

The present invention is illustrated by the following example. It is to be understood that the particular example, materials, amounts, and procedures are to be interpreted broadly in accordance with the scope and spirit of the invention as set forth herein.

### EXAMPLE

This example describes technology for manufacturing and characterization of oat derived, salt precipitated β-(1,3)-(1,4) glucan. In brief, oat bran derived, salt precipitated β-(1,3)-(1,4) glucan fractions with specifically defined Mw range can be obtained from the β-glucan concentrates using a technology that involves salt-based precipitants and that allows for a 10-liter scale production. The oat bran derived, salt precipitated β-(1,3)-(1,4) glucan with a MW of about 200 kDa (i.e., 200 kDa β-(1,3)-(1,4) glucan) were obtained and checked for purity, structure, potency and sterility. The results demonstrated that oat bran derived, salt precipitated β-(1,3)-(1,4) glucan is a highly purified (>95%), unmodified carbohydrate polymer which consists of (1,3) and (1,4)-β-glycosidic linkages. The oat bran derived, salt precipitated β-(1,3)-(1,4) glucan is completely soluble in phosphate-buffered saline (PBS), has a peak molecular weight (Mw) of about 200 kDa (Mw/Mn about 1.2), and is characterized by a DP3/DP4 ratio of about 1.5 to about 2.0.

The oat bran derived, salt precipitated 200 kDa β-(1,3)-(1,4) glucan was found to be free of bacterial, fungi and endotoxin. Since studies have demonstrated that the peptide/protein impurities, endotoxin contamination and broad Mw distribution could significantly affect biological activity mediated by β-glucan, the unique features of the 200 kDa β-(1,3)-(1,4) glucan make them excellent immune modulator candidates for clinical application and the regulatory approval.

Below is a discussion of the preparation, manufacturing, characterization and control of the 200 kDa β-(1,3)-(1,4) glucan using organic oat bran.

### Preparation of Starting Material (Organic Oat Brans)

### Use of Organic Oat Brans as Starting Material

β-(1,3)-(1,4)-glucan is found in the endosperm cell walls and in the subaleuone layer of cereals including oats, barley, wheat and rye. Barley and oat bran contain 2.8-15% of β-glucan whereas in wheat and rye the main component of soluble dietary fiber is arabinoxylan and the amounts of β-glucan are small. Soluble β-glucan levels are reported to be 50% higher in the oats than in the barley. Since soluble β-glucans have been reported to be the major components of dietary fibers mediating multiple health benefits, we selected organic oat bran as starting materials to produce the salt precipitated 200 kDa β-(1,3)-(1,4) glucan.

### Use Trackable Oat Bran as Starting Material

We used organic oat brans purchased from food market for 200 kDa β-(1,3)-(1,4) glucan manufacturing. In order to produce clinical supply of 200 kDa β-(1,3)-(1,4) glucan, we propose to use Oat Bran Flakes from Spectrum laboratory Products Inc. Spectrum lab provides catalog number (0103), trackable lot number and certificate of analysis for their oat bran products. Information that can be tracked to a specific lot includes:
Protein content (-17%)
Moisture (-12%)
Fat (9%)
Microbial limits: total plate count (˜10,000/g)
Yeast and mold (500/g)
Escherichia coli (negative)
Salmonella (negative)

### Preparation, Labeling and Storage of Starting Material

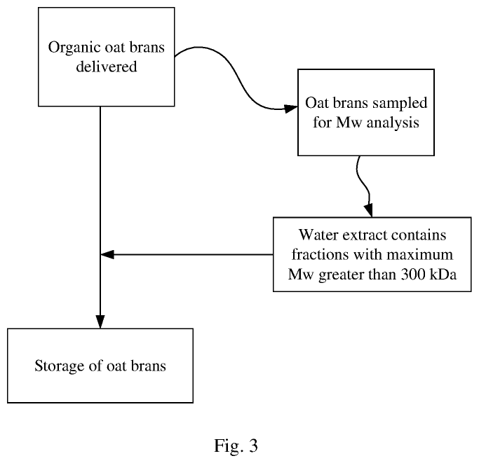

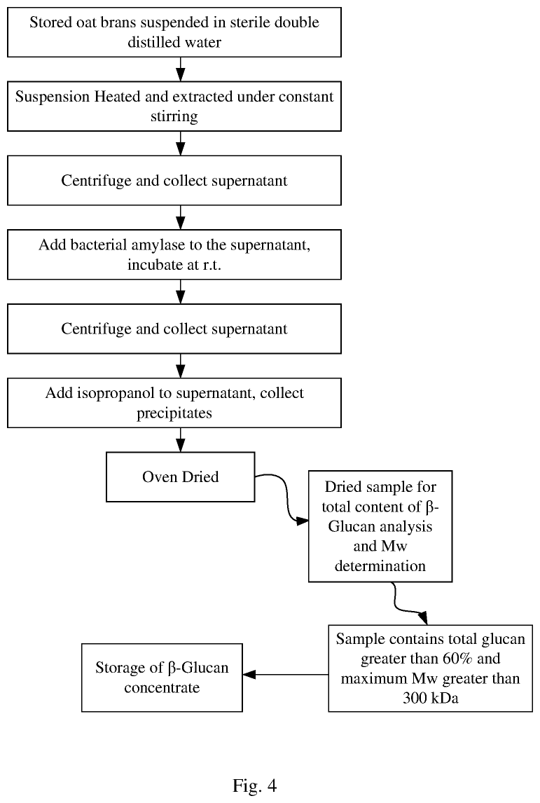

Referring to FIG. 3, once organic oat brans with trackable lot number are delivered, we can sample 1 gram and conduct a quick test to examine whether the hot water extract contains soluble fractions with maximum Mw greater than 300 kDa. One gram of O103 oat bran can be weighted and suspended in 200 mL double distilled water in autoclaved 500 mL glass beaker. The suspension can be boiled on a heating plate for 30 minutes with constant stirring and then stirred until the slurry cool down to room temperature (r.t.) (e.g., about 25° C.) Followed by centrifugation for 20 minutes at 8,000 rpm, the supernatant can be collected, 5× diluted with PBS, and then subjected to GPC-RI analysis to determine the minimum and maximum value of Mw contained in the sample. Starting material that contains soluble fractions with a maximum Mw greater than 300 kDa can be stored in an appropriately labeled clinical containers . The container can be stored at r.t. in the lab. The starting materials can be used for 200 kDa β-(1,3)-(1,4) glucan manufacturing within 30 days upon delivery.

### Preparation, Labeling and Storage of β-Glucan Concentrate

### Preparation of β-Glucan Concentrate

Referring to FIG. 4, 250 grams of qualified starting material can be weighted and suspended in 5 liters sterile double distilled water in clean and autoclaved beakers. The beakers can be heated in a water bath at 60° C. for 30 minutes under constant stirring and then stirred until the slurry cool down to room temperature. Followed by centrifugation for 8,000 rpm at 30 minutes, the supernatant can be collected and incubated with bacterial α-amylase at r.t. for 2 hours. After incubation, the slurry can be centrifuged at 8,000 rpm for 30 minutes to collect the supernatant. The supernatant can be precipitated by 60% isopropanol. Precipitates can be collected by decanting the supernatant. Precipitates can be oven-dried at 60° C.

### Total β-Glucan Content Analysis and Mw Determination

The oven-dried product can be sampled 5 mg to determine the total content of β-glucan using AOAC method. The oven-dried product can also be sampled 5 mg to determine Mw using GPC-RI analysis. Please refer to section 7.3.4 for test methods and controls. Weight percentage of total β-glucan content should be greater than 60% and the soluble product should contain fractions with maximum Mw greater than 300 kDa.

### Labeling and Storage

β-glucan concentrates prepared from the same lot of starting materials can be combined and stored in an appropriately labeled clinical grade containers. The container can be stored at 4° C. in the refrigerator in the lab. The combined β-glucan concentrates can be used for manufacturing the salt precipitated 200 kDa β-(1,3)-(1,4) glucan within 90 days upon the first log date.

### Salt Precipitated 200 kDa β-(1,3)-(1,4) Glucan Manufacturing

Referring to FIG. 5, 250 grams of β-glucan concentrate can be weighted and suspended in 10 liters sterile double distilled water in a 15-liter reactor. The suspension can be heated to 80° C., extracted at 80° C. for 60 minutes with constant stirring, and stir until the slurry cooled down to r.t. Followed by centrifugation for at 8,000 rpm 30 minutes, the supernatant can be collected. A mixture of salt precipitants can be slowly added to the supernatant to a final concentration of 15% (w/w %) under constant stirring. After addition, the slurry can be incubated at r.t. for 45-60 minutes with no stirring. Centrifuge the slurry at 8,000 rpm for 30 minutes, collect supernatant and precipitate. 10 ml supernatant can be sampled for QC Mw analysis. Maximum Mw contained in the supernatant should be smaller than about 220 kDa in order to move to the next step. If the maximum Mw contained in the supernatant is greater than 220 kDa, add additional precipitants to the supernatant to a final concentration of about 0.5% (w/w %) with constant stirring. After addition, the slurry can be incubated at r.t. for 45-60 minutes with no stirring. Centrifuge the slurry at 8,000 rpm for 30 minutes, collect supernatant and precipitate. 10 ml supernatant can be sampled for QC Mw analysis. If the maximum Mw is still greater than 220 kDa, repeat this process until the maximum Mw contained in the supernatant is smaller than 200 kda.

After this process is done, collect supernatant and add a mixture of salt precipitants to the supernatant to a final concentration of 20% (w/w %) with constant stirring. After addition, the slurry can be incubated at r.t. for 45-60 minutes with no stirring. Centrifuge the slurry at 8,000 rpm for 30 minutes, collect supernatant and precipitate. 10 ml supernatant can be sampled for QC Mw analysis. Maximum Mw contained in the supernatant should be smaller than 180 kDa in order to move to the next step. If the maximum Mw contained in the supernatant is greater than 180 kDa, add additional precipitants to the supernatant to a final concentration of about 0.5% (w/w %) with constant stirring, place at r.t. for 45-60 minutes, centrifuge, collect supernatant for QC Mw analysis, and repeat this process until the maximum Mw contained in the supernatant is smaller than 180 kDa. After this process is done, collect precipitates, dissolve the precipitates in sterile double distilled water, and dialyze the solution against sterile double distilled water over night. Add isopropanol to the dialyzed solution at a final concentration about 50% (v/v %) to precipitate the glucan. Collect the precipitates by decanting the supernatant. The precipitates can be washed twice using 60%, 75%, and 95%, respectively and sequentially. The precipitates in 95% isopropanol can be centrifuged to remove the supernatant. Such obtained precipitates can be oven-dried at 60° C.

### Salt Precipitated 200 kDa β-(1,3)-(1,4) Glucan Characterization

One gram of oven-dried product can be sampled for characterization using proper controls. Characterization includes:
GPC-NMR analysis to determine the chemical structure, Mw and Mw/Mn (molecular weight distribution)
DP3/DP4 ratio as indicator of structural differences
Purity analysis using AOAC methods to determine the total content of carbohydrate glucan, protein, fat/oil, moisture and ashes
Sterility test to examine whether there is bacteria or fungi contamination
Endotoxin assay
Potency assay using cell-based studies

### Description of Test Methods

### Purity Assay

Purity of the salt precipitated 200 kDa ⊕-(1,3)-(1,4) glucan product was determined by analyzing the total content of β-glucan, starch, protein, fat/oil, moisture and ashes. Methods and standard protocols of these analyses are well established in food industry. These assay procedures were verified by processing and measuring two standard samples with known content of components (Standard glucan samples from Megazyme, Ireland). Results demonstrated that the salt precipitated 200 kDa β-(1,3)-(1,4) glucan produced by our manufacturing was a highly-purified product with β-Glucan content over 95% (Table 2).

TABLE 2
Purity Assay of the salt precipitated 200 kDa β-(1,3)-(1,4) glucan

| Description | Kits | Method | Results |
|---|---|---|---|
| Total glucan | Mixed-Linkage β-Glucan | AOAC Method | >95% |
| content | Assay Kit (Megazyme, | 32-23.01 and | |
| | Ireland) | 995.16. | |
| Starch | Total Starch Assay Procedure | AOAC method | Not |
| | Kit (Megazyme, Ireland) | 995.16 | detected |
| Protein | n.a | AOAC method | ~1% |
| | | 991.20 | |
| Fat/oil | n.a. | AOAC method | Not |
| | | 991.20 | detected |
| Moisture | n.a. | Gravimetric | ~3% |
| | | Analysis | |
| Ashes | n.a. | Corn Refiners | ~1% |
| | | Association | |
| | | Standard | |
| | | Analytical | |
| | | Method A-4 | |

Determination of Chemical Structure, Mw and Mw/Mn (Molecular weight distribution)

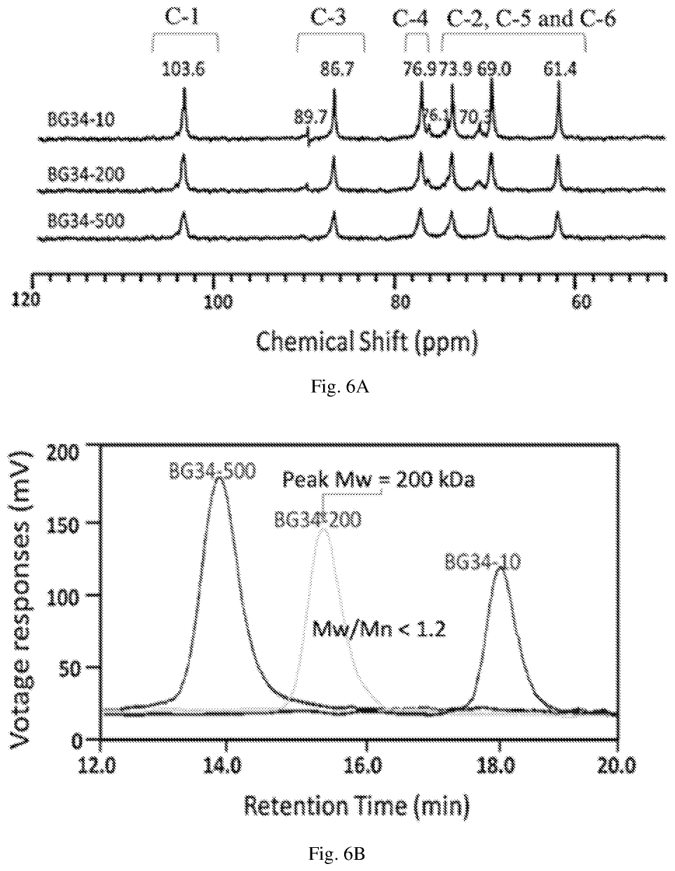

We have conducted high resolution ¹³C NMR analysis of salt precipitated β-(1,3)-(1,4) glucan of three different molecular weight. Signals were recorded in DMSO at 500.13 MHz on a burker AM500 NMR spectrometer operating at 80° C. The chemical shifts were referenced to DMSO-d6 at ˜39 ppm for ¹³C and are reported relative to TMS. The ¹³C spectroscopy and experiments were run using the standard Bruker pulse sequence. ¹³C NMR analysis of cereal-derived β-(1,3)-(1,4)-glucans have been well documented in literature. Our spectrum of the salt precipitated 200 kDa β-(1,3)-(1,4) glucan appeared identical to that of the β-(1,3)-(1,4)-glucans isolated from oat or barley. Chemical shifts at ˜103.6 ppm, 86.7-89.7 ppm and 76.1-76.9 ppm were assigned to carbon 1 (C-1), C-3 and C-4, indicating the β-(1-3)- and β-(1-4)-glycosylic linkages (FIG. 6A).

The Mw and Mw/Mn of the salt precipitated 200 kDa β-(1,3)-(1,4) glucan were determined by Gel Permeation Chromatograph (GPC) using two columns in series (Shodex Ohpak KB-806M, Showa Denko K.K., Tokyo, Japan; Ultrahydrogel linear, Waters, Milford, USA). GPC system was connected with Refractive Index (RI) Detectors that allowed the determination of Mw and Mw/Mn. Salt precipitated 200 kDa β-(1,3)-(1,4) glucan samples in PBS at about 1 mg/ml were filtered (0.45 pm) and injected 100 μL to the system. The system was eluted with 0.1 M NaNO₃ (0.6 ml/min) at 37° C. Determination of Mw and Mw/Mn were obtained by using a calibration using β-glucan Mw Standard (Megazyme, Catalog number: P-MWBGS) that contains β-(1,3)-(1,4)-glucans with seven different molecular weights. Results demonstrate the peak Mw of 200 kDa and Mw/Mn<1.2 for the product (FIG. 6B)

### DP3/DP4 Ratio

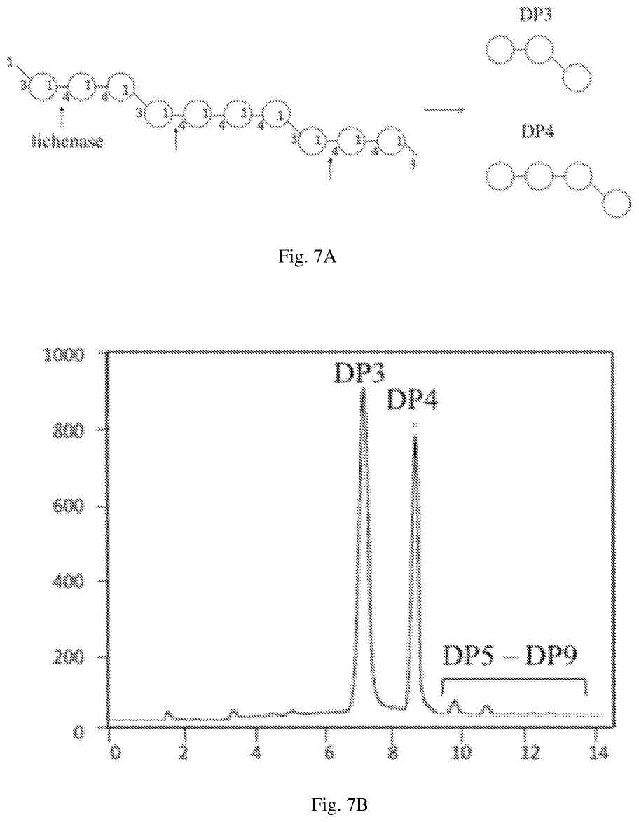

DP3/DP4 is an often-used indicator of structural differences of cereal derived β-(1,3)-(1,4)-glucans. It has been documented in literature that cereal derived β-glucans are linear polysaccharide that consist only of β-D-glucopyranosyl unit. These units are joined by either (1,3)- or (1,4)-β-D linkages. Structure sequence analysis if often carried out by breaking the (1,4) linkage next to a (1,3) linkage at the reducing end using enzyme lichenase (EC 3.2.1.73). This results in the generation of (1,4)-linked oligosaccharides with (1,3)-linked glucose unit as an end group at the reducing end. The oligosaccharide with degree of polymerization of 3 (DP3), i.e., 3-O-β-cellobiosyl-D-glucose, is the main product, the oligosaccharide DP4, i.e., 3-O-β-cellotriosyl-D-glucose, comes second. These two building blocks, DP3 and DP4, constitute over 90% of the molecule. The remaining oligosaccharides contain longer sequences. Oligosaccharides of up to DP13 are found in cereal derived soluble β-glucans, and oligosaccharides of up to DP20 are reported in the insoluble β-glucans. (FIG. 7)

Importantly, DP3/DP4 ratio is used as indicator of solubility. The ratio is higher for insoluble than for soluble β-glucans. Studies have shown that the more there are (1,3)-linkages in a molecule, the greater is the possibility of consecutive cellotriosyl units (DP3). These DP3 units can form helices and cause insolubility through aggregation, thus to impact the biological activity. Wood et al. reported values 2.1-2.4 for soluble oat β-glucans, 2.8-3.3 for barley β-glucans and 3.0-3.2 for rye β-glucans. Izydorczyk et al. obtained values 1.76 and 2.13 for soluble barley β-glucan extracted at 40° C. and 65° C., respectively and for insoluble β-glucan they reported values of 2.07-2.43. For wheat β-glucan the ratio is 3.1-4.5.

We have conducted HPAEC-PAD analysis to determine DP3/DP4 ratio after hydrolyzing the salt precipitated 200 kDa β-(1,3)-(1,4) glucan using lichenase using an AOAC method (AOAC 995.15). After lichenase treatment, the oligosaccharides produced from the treatment were filtered through 0.2 μm filter and loaded for the HPAEC-PAD analysis. The system was eluted with gradient eluents. The eluents contained A (150 mM NaOH) and B (500 mM sodium acetate in 150 mM NaOH), and the gradient was changed from 85% A and 15% B to 100% B in 20 minutes. Quantification analysis was performed by using malto-oligosaccharides with DP 3-12 (Sigma). Results of HPAEC-PAD analysis demonstrated that the DP3/DP4 is about 1.5 to about 2.0 for the salt precipitated 200 kDa β-(1,3)-(1,4) glucan (FIG. 7).

### Potency Assay

We have conducted cellular assay to test the biological activity of the salt precipitated 200 kDa β-(1,3)-(1,4) glucan in vitro. In vitro studies using THP1 cell model have also revealed that the biological activity is significantly affected by Mw characteristic of the salt precipitated β-(1,3)-(1,4) glucan. THP1 cells are immortalized human monocyte cell line that can be differentiated and matured into M1 activated or M2 activated macrophages or DCs in response to external stimulus during the culture. THP1 cell culture in the absence and presence of salt precipitated β-(1,3)-(1,4) glucan molecules of different Mw have demonstrated that salt precipitated β-(1,3)-(1,4) glucan in the range of 100 and 300 kDa can effectively modulate THP1 differentiation into cells with DC-like phenotype and function.

Cell studies using human monocyte THP-1 cell line revealed that the salt precipitated 200 kDa β-(1,3)-(1,4) glucan exposure could modulate the monocyte differentiation into cells with DC-like phenotype and function with significantly upregulated activation markers (CD80, CD86, MHC II and CD11c), increased production of inflammatory cytokines (TNF-α and IL-12) and enhanced phagocytosis.

Based upon these results, we propose to use THP1 cell model and analysis for potency assay of clinical supply of the salt precipitated 200 kDa β-(1,3)-(1,4) glucan. The phenotypical and functional alteration of THP1 cells upon the salt precipitated 200 kDa β-(1,3)-(1,4) glucan treatment can be characterized by measuring intracellular production of TNF-c and IL-12 using quantitative ELISA, and by quantifying the cell surface expression of MHC II using FACS analysis.

### Sterility Test—USP

We have conducted sterility test of the salt precipitated 200 kDa β-(1,3)-(1,4) glucan solution before the salt precipitated 200 kDa β-(1,3)-(1,4) glucan was administered to mice. The sterility test was to determine if the salt precipitated 200 kDa β-(1,3)-(1,4) glucan solution is free from viable bacterial and fungal contamination. The salt precipitated 200 kDa β-(1,3)-(1,4) glucan was dissolved in sterile PBS solution at 1 mg/mL and filtered through 0.45 pm filter. The salt precipitated 200 kDa β-(1,3)-(1,4) glucan solution was aseptically transferred into Soybean-Casein Digest Medium (SCDM) and Fluid Thioglycollate Medium (FTM). These broths were incubated for 14 days and inspected for evidence of bacterial and fungal growth. Results indicated that bacteria and fungi were not detected in salt precipitated 200 kDa β-(1,3)-(1,4) glucan samples (data not shown). The sterility test of salt precipitated 200 kDa β-(1,3)-(1,4) glucan was performed at UHCMC Microbiology Lab.

### BET/LAL Kinetic/ChromI/E Assay—USP/FDA

We have conducted this assay which validates the use of the LAL gel-clot or kinetic-chromogenic method for the detection of endotoxins. Validation requires inhibition/enhancement (I/E) testing and determination of endotoxin levels in at least three product lots. Testing must be performed on the undiluted product or on an appropriate dilution of the final product, and results must be within the prescribed endotoxin limits. The potential for interference by the test product was examined by spiking the salt precipitated 200 kDa β-(1,3)-(1,4) glucan solution (1 mg/mL in PBS) plus LAL reagent with specified levels of endotoxin. Following the inhibition/enhancement test, the endotoxin content of the salt precipitated 200 kDa β-(1,3)-(1,4) glucan was determined. The results indicated that the endotoxin level was <5 EU/kg for the total product dose in mice (data not shown). This assay was conducted at the cGMP facility of CWRU/UHCMC Cellular Therapy Laboratory.

### Preparation of IV Infusion Bag

The final salt precipitated 200 kDa β-(1,3)-(1,4) glucan drug product, consists of the salt precipitated 200 kDa β-(1,3)-(1,4) glucan molecules suspended in sterile, injectable PBS at a concentration of 2 ±0.1 mg/mL in clinical grade sterile drug vials at 10 ml/vial. The liquid pharmaceutical composition can be tested for sterility using gram stain (bacteria), mycoplasma (qPCR) and endotoxin testing (limulus assay) which can be used as release criteria administration of the final drug product. The drug vials containing the liquid pharmaceutical composition of the salt precipitated 200 kDa β-(1,3)-(1,4) glucan can be stored at r.t. during the sterility test mentioned as above. The salt precipitated 200 kDa β-(1,3)-(1,4) glucan solution that meets the final release criteria can be directly added to IV infusion bag that contained a certain volume of injectable PBS. The final formulation in the IV solution bag can consist of salt precipitated 200 kDa β-(1,3)-(1,4) glucan at 0.2±0.01 mg/mL in injectable PBS. Total amount to be added to the infusion bag is calculated based on patients' body weight. The final formulation housed within sterile IV solution infusion bag can be stored at r.t. Each infusion bag can be labeled appropriately to allow for confirmation of patient identity prior to infusion.

### Sterility Test—USP

This procedure is used to determine if the test article is free from viable bacterial and fungal contamination. The test article is aseptically transferred into Soybean-Casein Digest Medium (SCDM) and Fluid Thioglycollate Medium (FTM). These broths are incubated for 14 days and inspected for evidence of bacterial and fungal growth.

### Bacteriostasis/Fungistasis (B/F)—USP

The B/F test is a validation of the sterility test and is performed to ensure that if viable bacteria or fungi were present, they would be apparent. Sterility test broths containing the test article are inoculated with low levels of specified microorganism and then inspected for evidence of microbial growth. Growth indicates no bacteriostatic or fungistatic activity and means the sterility test parameters are valid.

### Gram Stain

Gram stain is a validation of the sterility test and is performed to visualize the presence of viable bacteria or fungi within the final infusion product.

### Mycoplasma Detection—Rapid

For the purpose of identifying the presence of mycoplasma contamination prior to the day of infusion, a sample of the salt precipitated 200 kDa β-(1,3)-(1,4) glucan solution can undergo rapid testing using either PCR or ELISA based methodology.

### Mycoplasma Detection—Culture

This assay is used to detect the presence of mycoplasma by both indirect (cell culture) and direct (broth and agar) assays. The test article is incubated with monkey kidney cells and in then fixed, stained with a DNA-binding fluorochrome (Hoechst Stain), and evaluated microscopically by epifluorescence for the presence of mycoplasma. Agar plates and broth flasks are inoculated with the test article and are incubated anaerobically and aerobically, respectively. Samples from the broth flasks are subcultured on day 3, 7 and 14 onto agar plates; all plates are examined no sooner than 14 days post-inoculation. These species of mycoplasma serve as positive controls.

### Drug Product Storage, Tracking and Labeling

Drug product that meets the product specification can be directly added to IV infusion bag solution bag that contains a certain volume of injectable PBS. The final formulation in the IV solution bag can consist of the salt precipitated 200 kDa β-(1,3)-(1,4) glucan at 0.2±0.01 mg/mL. Total amount of solution to be added to the infusion bag is calculated based on patients' body weight. The final formulation housed within sterile IV solution infusion bag can be stored at r.t.

### Stability Test

It has been well documented that soluble oat derived β-(1,3)-(1,4)-glucans do not spontaneously form aggregates, organized structures or patterns in PBS solution. They are not sensitive to pH, light, temperature, mechanical force, electric and magnetic fields. Because of that, we chose to test the stability of the salt precipitated 200 kDa β-(1,3)-(1,4) glucan by determining the Mw in PBS solution in 6 months. The salt precipitated 200 kDa β-(1,3)-(1,4) glucan was dissolved in PBS at 1 mg/mL and filtered through 0.45 μm filter, which was conducted in aseptic hood. Such the prepared the salt precipitated 200 kDa β-(1,3)-(1,4) glucan solution was stored in sterile drug vials and placed at r.t. At different time points, 100 μL of the salt precipitated 200 kDa β-(1,3)-(1,4) glucan solution was pipetted out aseptically for Mw determination. The results of stability testing of three independent salt precipitated 200 kDa β-(1,3)-(1,4) glucan samples manufactured in our lab are shown in Table 3. Mw and Mw/Mn of samples were determined by GPC-RI analysis. Potency of these three salt precipitated 200 kDa β-(1,3)-(1,4) glucan samples were determined on day 0 and day 180. Potency was determined by quantifying the THP1 cell production of TNF-α upon exposure to the salt precipitated 200 kDa β-(1,3)-(1,4) glucan for 72 hours, using PBS as control. These results indicated that Mw and Mw/Mn of the final product did not undergo any significant change over 6 month; the salt precipitated 200 kDa β-(1,3)-(1,4) glucan induced activity (cell production of TNF-c) remained over 90% after 6 month of storage at r.t

TABLE 3
salt precipitated 200 kDa β-(1,3)-(1,4)
glucan molecular weight Stability Test Results

| | 0 Day | | | 14 Days | | |
|---|---|---|---|---|---|---|
| Sample ID | #1 | #2 | #3 | #1 | #2 | #3 |
| Mw | 200 ± 5 | 198 ± 6 | 203 ± 4 | 199 ± 7 | 198 ± 3 | 201 ± 6 |
| (peak) | Kda | kDa | kDa | kDa | kda | kDa |
| Mw/Mn | 1.17 | 1.20 | 1.15 | 1.17 | 1.21 | 1.15 |
| | 30 days | | | 60 day | | |
| Sample ID | #1 | #2 | #3 | #1 | #2 | #3 |
| Mw | 197 ± 5 | 200 ± 3 | 203 ± 6 | 201 ± 4 | 198 ± 5 | 201 ± 2 |
| (peak) | kDa | kDa | kDa | kDa | kDa | kDa |
| Mw/Mn | 1.16 | 1.19 | 1.16 | 1.17 | 1.20 | 1.13 |
| | 90 days | | | 120 Days | | |
| Sample ID | #1 | #2 | #3 | #1 | #2 | #3 |
| Mw | 202 ± 5 | 201 ± 6 | 204 ± 3 | 205 ± 3 | 204 ± 5 | 203 ± 4 |
| (peak) | kDa | kDa | kDa | kDa | kDa | kDa |
| Mw/Mn | 1.22 | 1.23 | 1.22 | 1.20 | 1.21 | 1.23 |

| | 180 Days | | |
|---|---|---|---|
| Sample ID | #1 | #2 | #3 |
| Mw | 197 ± 6 | 196 ± 7 | 200 ± 5 |
| (peak) | kDa | kDa | kDa |
| Mw/Mn | 1.19 | 1.18 | 1.17 1 |

While this invention has been particularly shown and described with references to preferred embodiments thereof, it can be understood by those skilled in the art that various changes in form and details may be made therein without departing from the scope of the invention encompassed by the appended claims. All patents, publications and references cited in the foregoing specification are herein incorporated by reference in their entirety.

1. A composition comprising;
an oat derived, salt precipitated β-(1,3)-(1,4) glucan having a molecular weight from about 150 kDa to about 250 kDa, a Mw/Mn ratio of about 1.0 to about 1.25, and a D3/D4 ratio of about 1.5 to less than about 2.0.

2. The composition of claim 1, comprising at least about 92% or more by weight of the oat derived, salt precipitated β-(1,3)-(1,4) glucan.

3. The composition of claim 1, comprising less than about 2% by weight protein and less than 0.001% by weight fat.

4. The composition of claim 1, wherein the oat derived, salt precipitated β-(1,3)-(1,4) glucan upon administration to a THP1 cell culture modulates THP1 cells into cells with DC like phenotype characterized by upregulated activation markers CD80 CD86, MHC II, and CD11c and increased production of inflammatory cytokines TNF-α and IL-12, and enhanced phagocytosis.

5. The composition of claim 1 having an endotoxin level less than about 5 EU/kg.

6. A pharmaceutical composition comprising an oat derived, salt precipitated β-(1,3)-(1,4) glucan of claim 1, and a pharmaceutically acceptable carrier.

7. The pharmaceutical composition of claim 6, wherein the pharmaceutically acceptable carrier includes phosphate buffered saline LPBSI.

8. The pharmaceutical composition of claim 6, comprising an injectable solution that includes about 1 mg/ml to about 5 mg/ml of the oat derived, salt precipitated β-(1,3)-(1,4) glucan.

9. A method of producing an oat derived, salt precipitated β-(1,3)-(1,4) glucan, the method comprising:
providing an aqueous suspension of β-glucan oat bran concentrate,
fractional precipitating an aqueous extract of the aqueous suspension with at least one salt precipitant to obtain an oat derived, precipitated β-(1,3)-(1,4) glucan having a molecular weight from about 150 kDa to about 250 kDa, a Mw/Mn ratio of about 1.0 to about 1.25, and a D3/D4 ratio of about 1.5 to less than about 2.0.

10. The method of claim 9, wherein the wherein the β-glucan oat bran concentrate has glucan concentration of at least 60% by weight.

11. The method of claim 9, wherein the at least one salt precipitant includes a mixture of salts.

12. The method of claim 11, wherein the mixture of salts includes ammonium sulfate.

13. The method of claim 9, wherein the aqueous extract is provided by heating the aqueous suspension to about 75° C. to about 90° C. for a duration of time effective dissolve soluble β-glucan in the aqueous suspension, cooling the aqueous suspension, and collecting the aqueous extract of the β-glucan oat bran concentrate from the cooled aqueous suspension.

14. The method of claim 9, wherein the fractional precipitation includes two or more fractionation steps.

15. The method of claim 14, wherein the fractional precipitation includes a first fractionation step, the first fractionation step including adding a first salt precipitant to the aqueous extract of the aqueous suspension at a concentration effective to form a first precipitate and a first supernatant and separating the first supernatant and the first precipitate, wherein the first supernatant has a maximum γ-glucan molecular weight less than about 230 kDa, less than about 225 kDa, less than about 220 kDa, or less than about 215 kDa.

16. The method of claim 15, wherein the fractional precipitation includes a second fractionation step, the second fractionation step including adding a second salt precipitant to the separated first supernatant at a concentration effective to form a second precipitate and a second supernatant and separating the second precipitate ad the second supernatant, wherein the second supernatant has a maximum β-glucan molecular weight less than about 175 kDa, less than about 180 kDa, less than about 185 kDa, or less than about 190 kDa.

17. The method of claim 16, further comprising forming an aqueous solution of the second precipitate and dialyzing the aqueous solution of the second precipitate to remove residual first salt precipitant and second salt precipitant from the aqueous solution of the second precipitate.

18. The method of claim 17, further comprising adding an alcohol to the dialyzed aqueous solution to precipitate the oat derived, precipitated β-(1,3)-(1,4) glucan having a molecular weight from about 150 kDa to about 250 kDa, a Mw/Mn ration of about 1.0 to about 1.25, and a D3/D4 ratio of about 1.5 to less than about 2.0.

19. The method of claim 16, wherein the first salt precipitant has the same composition as the second salt precipitant.

20. The method of claim 19, wherein the first salt precipitant and the second salt precipitant include a mixture of salts, wherein at least one of the salts in the mixture is ammonium sulphate.

21. The method of claim 20, wherein the first salt precipitant is added to the first supernatant at a w/w % that differs from a w/w % at which the second salt precipitant is added to the second supernatant.

22-28. (canceled)
